(19) United States
(12) Patent Application Publication
(10) Pub. No.: US 2026/0346436 A1
(43) Pub. Date: Nov. 5, 2026

(54) Distributed Learning and Inter-Cobot Communication for Enhanced Perioperative Functionality

(71) Applicant: Peritas AI Inc., Fort Lauderdale, FL (US)

(72) Inventor: Martin Roche, Fort Lauderdale, FL (US)

(73) Assignee: Peritas AI Inc., Fort Lauderdale, FL (US)

(21) Appl. No.: 19/428,294

(22) Filed: Dec. 21, 2025

Related U.S. Application Data
(63) Continuation-in-part of application No. 19/195,731, filed on Apr. 30, 2025.

(51) Int. Cl. B25J 9/16; A61B 34/32; B25J 13/00; G05B 13/02; G16H 10/60

(52) U.S. Cl. CPC B25J 9/163; A61B 34/32; B25J 9/1682; B25J 13/006; G05B 13/0265; G16H 10/60

(57) ABSTRACT

A networked cobotic system is disclosed for enhancing perioperative functionality through distributed learning and inter-cobot communication. The system comprises multiple autonomous cobots configured to collaborate within surgical environments, each equipped with sensor suites, wireless communication interfaces, and onboard control systems. A distributed machine learning framework enables local model training based on perioperative data and parameter sharing among cobots, facilitating continuous system-wide learning while preserving data privacy through federated methods. A real-time communication interface supports dynamic task coordination, sensor data exchange, and unified environmental perception. An adaptive task allocation engine assigns responsibilities based on situational analysis, predictive models, and system state. The system further integrates a collective decision-making module responsive to environmental changes and procedural contingencies, and a self-organizing safety protocol ensuring compliance with sterile field boundaries and surgical safety standards. A centralized visualization interface provides real-time oversight, system status, and predictive analytics to perioperative staff.

### CROSS-REFERENCE TO RELATED APPLICATIONS

This patent application is a continuation-in-part of US Patent Application Ser. No. 19/195,731 filed on Apr. 30, 2025, which claims the benefit of priority to U.S. Provisional Patent Application No. 63/640,524, filed on Apr. 30, 2024, the entirety of which is incorporated herein by refer

### TECHNICAL FIELD

The present invention relates generally to collaborative robotic systems in surgical and perioperative environments, and more specifically to networked cobotic systems implementing distributed machine learning and inter-cobot communication.

### BACKGROUND

Perioperative workflows in modern surgical settings are increasingly complex and resource-intensive, often requiring dynamic coordination across surgical teams, equipment, and support infrastructure. Robotic systems have become integral to many perioperative environments, offering capabilities such as instrumentation handling, environmental monitoring, and autonomous navigation. While individual cobots have demonstrated utility in supporting specific tasks, their ability to scale across broader perioperative operations remains limited by their reliance on siloed control systems and static task assignments.

Current robotic systems typically operate in isolation or depend on centralized command-and-control architectures. These approaches introduce performance bottlenecks, reduce fault tolerance, and lack the responsiveness required to adapt to rapidly changing perioperative conditions. Additionally, the data generated by individual cobots, ranging from sensor data to task execution outcomes, remains underutilized due to the absence of collaborative data processing frameworks.

There is a growing need for cobotic systems that not only operate autonomously but also communicate and collaborate in real time. Advances in federated learning and distributed machine learning have opened pathways for such systems to improve continuously while preserving data locality and patient privacy. However, integration of these technologies into surgical environments poses challenges related to latency, data security, sterile compliance, and task coordination under variable constraints.

Moreover, effective deployment of multiple cobots in a shared clinical environment requires robust mechanisms for task allocation, inter-device communication, environmental perception, and safety compliance. Without a unified strategy for coordination and learning, multi-cobot deployments risk redundancy, conflict, and reduced overall efficiency.

Accordingly, there exists a need for a networked cobotic system configured for distributed intelligence and real-time inter-cobot communication that enables collaborative decision-making, adaptive task distribution, and continuous learning, while maintaining compliance with safety protocols and privacy requirements in perioperative settings.

### SUMMARY

Embodiments described herein relate to a networked cobotic system for use in perioperative environments, in which a plurality of collaborative robotic units (cobots) are configured for autonomous operation and inter-cobot communication. Each cobot is equipped with a control system, a sensor suite, and a real-time communication interface, enabling it to operate independently while participating in coordinated actions with other cobots in the network .

The system implements a distributed machine learning framework wherein cobots locally train models based on perioperative data acquired during operation and exchange learned parameters with peer units. This architecture enables continual learning and performance enhancement across an agentic system without centralized processing bottlenecks or transmission of sensitive patient data.

A real-time inter-cobot communication interface facilitates high-speed, low-latency wireless data exchange among cobots, supporting collaborative functions such as task coordination, movement synchronization, sensor fusion, and unified environmental mapping. A collaborative task allocation system dynamically assigns surgical support tasks among the cobots based on current situational assessments, system state, and predictive analytics.

The system further includes federated data processing capabilities to preserve data privacy, as well as an adaptive collective decision-making engine that enables cobots to respond as a group to changes in the perioperative environment. Safety and compliance protocols are enforced through continuous self-organization among cobots, ensuring obstacle avoidance, sterile field adherence, and real-time monitoring of environmental conditions.

A centralized visualization and monitoring interface provides perioperative personnel with real-time insight into system activity, task status, performance metrics, and alerts, enabling efficient oversight and coordination.

### BRIEF DESCRIPTION OF DRAWINGS

The foregoing and other objects, aspects, features, and advantages of the disclosure will become more apparent and better understood by referring to the following description taken in conjunction with the accompanying drawings, in which:

FIG. 1 is a perspective view of an exemplary perioperative cobot.

FIG. 2 is a schematic block diagram illustrating the architecture of a single cobot unit.

FIG. 3 is a diagram of a networked cobotic system comprising multiple cobots.

FIG. 4 is a flowchart depicting the collaborative task allocation process among multiple cobots in response to real-time operational data.

FIG. 5 is a schematic representation of the distributed machine learning framework.

FIG. 6 is a block diagram showing the centralized visualization and monitoring interface for perioperative personnel.

FIG. 7 is a flowchart illustrating the adaptive collective decision-making logic.

FIG. 8 is a diagram of the safety and compliance enforcement subsystem.

These drawings are provided for illustrative purposes only and should not be construed as limiting the invention to the specific embodiments shown and described herein. Variations and modifications to these embodiments will be apparent to those skilled in the art, and all such variations and modifications are intended to be included within the scope of this disclosure.

### DETAILED DESCRIPTION

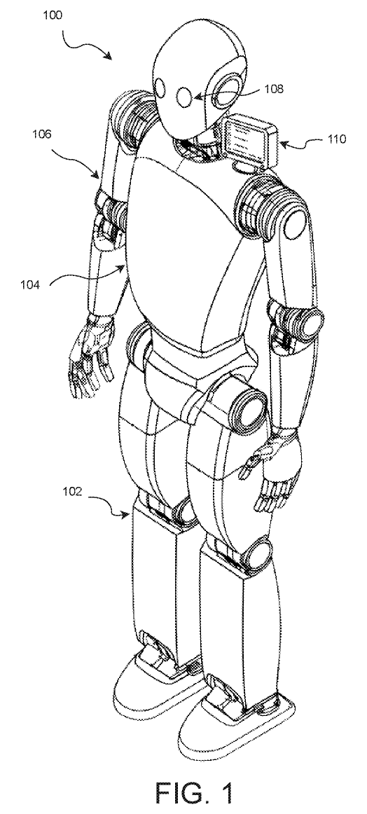

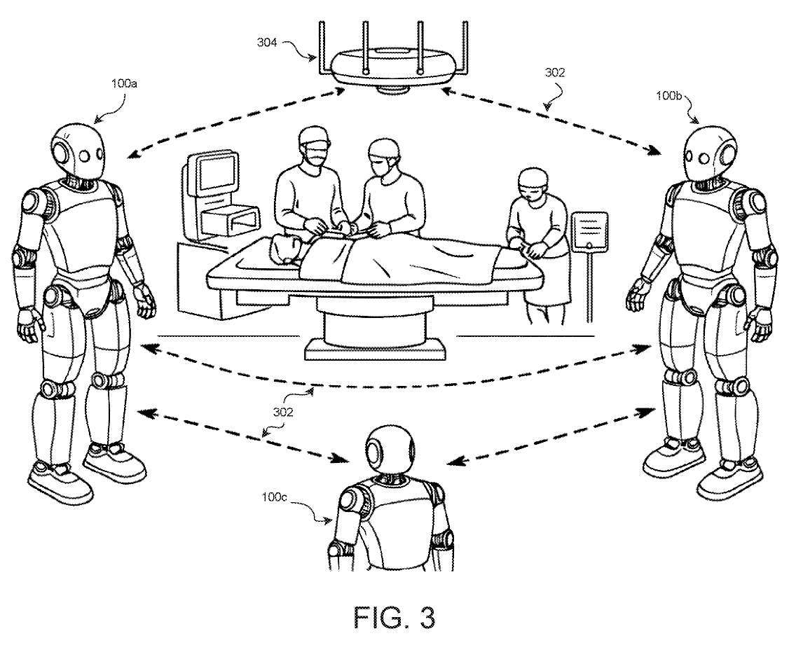

Referring now to the drawings, which illustrate an exemplary embodiment of the system and are not intended to limit the scope of the invention, FIG. 1 shows a perspective view of an exemplary perioperative cobot 100 configured for collaborative operation in a surgical environment. Cobot 100 includes a mobile base 102 supporting an upright body or mast 104 and one or more robotic arms or manipulators 106 for interacting with surgical instruments and the environment. The mobile base 102 may incorporate omnidirectional wheels or treads and motors that enable the cobot to autonomously navigate within an operating room. Mounted on the mast 104 and the arm 106 are various sensors, such as high-resolution cameras 108, depth sensors (e.g., LIDAR or structured light), and potentially biosensors or infrared detectors, which collectively provide the cobot with a rich situational awareness of its surroundings. The cobot 100 can also include a user interface panel or display 110 and status indicator lights to convey information to human staff (for instance, showing its mode or alerts), as well as an antenna or wireless transceiver (not shown) to facilitate communication with other devices. The mechanical design of cobot 100 emphasizes safe, collaborative use: for example, the arm 106 is equipped with force-limited actuators and soft padding or rounded edges to allow close human interaction without risk of injury. In operation, a single cobot 100 can perform useful perioperative tasks (such as fetching supplies or monitoring sterile fields) on its own, and multiple such cobots can be deployed simultaneously to work in concert (as described further with respect to FIG. 3).

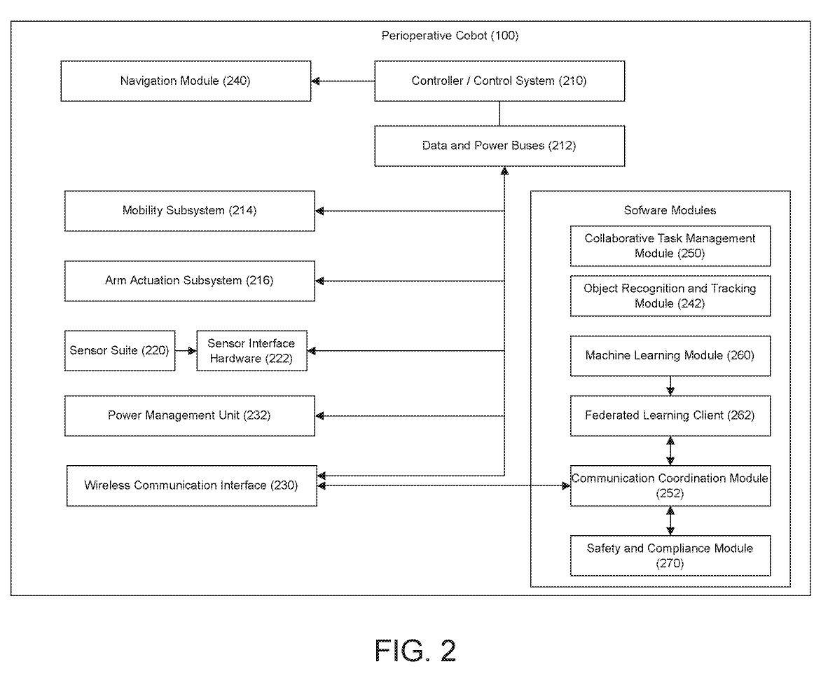

FIG. 2 is a schematic block diagram illustrating the internal architecture of the perioperative cobot 100. As shown, each cobot 100 is built around a control system 210 that includes one or more processors and memory devices programmed to control the cobot’s functions. The control system connects to a variety of subsystems via data and power buses 212. A mobility subsystem 214 comprises the drive motors, wheel actuators or leg actuators (in embodiments where the cobot can change locomotion mode), and associated motor controllers that enable movement and positioning of the base 102. An arm actuation subsystem 216 controls any robotic manipulator 106, including joint motors and end-effector actuators for gripping or tool manipulation. A suite of sensors 220 (corresponding to the cameras 108, depth sensors, environmental sensors, etc. on the cobot) interfaces with the controller 210 through sensor interface hardware 222, providing real-time data about the cobot’s environment, the location of surgical personnel, instrument identification, bioburden levels, and other contextual information. The cobot 100 also includes a wireless communication interface 230 (e.g., a Wi-Fi, BLE, or dedicated RF module with an antenna that enables low-latency, high-speed data exchange with other cobots and with any central server or network infrastructure. In addition, a power management unit 232 (such as a battery system and power regulators) supplies energy for untethered operation and can monitor battery levels and docking/charging status. The various hardware components are operably linked such that the controller 210 can receive inputs from sensors and other cobots and send commands to the motors and output devices in a coordinated manner.

On the software and functional level, the architecture of FIG. 2 further illustrates several key modules implemented by the controller 210 (in hardware, software, or firmware) to realize the cobot’s intelligent collaborative behavior. A navigation module 240 processes sensor data (for example, from depth sensors and cameras) to perform simultaneous localization and mapping (SLAM), obstacle detection, and path planning, allowing the cobot to move autonomously around equipment and personnel while avoiding collisions. An object recognition and tracking module 242 leverages machine vision (potentially using trained neural networks) to identify surgical instruments, track their usage, recognize staff members and their gestures, and monitor regions (such as a surgical site or sterile field boundaries) for events of interest. Integrated with these is a collaborative task management module 250, which is responsible for handling task assignments and coordination: this module evaluates incoming task requests (either from a central coordinator, from other cobots, or generated autonomously), determines the cobot’s availability and suitability, and communicates with the team (via the interface 230) to accept or delegate tasks as needed. The communication interface 230 is governed by a communication coordination module 252 that implements the inter-cobot communication protocols, ensuring that messages (like status updates, sensor data, or alerts) are packaged and transmitted reliably with minimal latency, and that incoming messages from other cobots are parsed and delivered to the appropriate functional modules. A machine learning module 260 is also incorporated, providing on-board data processing and learning capabilities: this may include local model training using the cobot’s collected data (e.g., refining a model for recognizing surgical phases or predicting instrument demand) and running inference for real-time decision support. The machine learning module 260 works in tandem with a federated learning client 262 that allows the cobot to share learned model updates with other cobots or a central aggregation service without exposing raw sensitive data. Finally, a safety and compliance module 270 continuously monitors the cobot’s operations and sensor inputs to enforce safety rules and protocol compliance. The safety module 270 can, for instance, establish virtual safety zones (such as the sterile field region the cobot must not enter with its base), regulate speeds and force outputs of the motors, and override or pause motions if a potential safety risk is detected. It also communicates with the communication module 252 so that safety-related information (like an emergency stop or a warning of a human too close to the cobot’s path) is broadcast to the other units. All of these subsystems and modules in FIG. 2 interoperate under the control of the controller 210, thereby equipping each cobot 100 to function independently with robust autonomous capabilities and to collaborate as part of a networked team.

Turning to the multi-cobot system level, FIG. 3 illustrates a networked of cobotic systems showing multiple perioperative cobots communicating and coordinating with each other. In the example depicted, a set of cobots 100a, 100b, 100c are deployed within an operating room or perioperative complex and are linked via a wireless network 302. This inter-cobot communication link 302 may be facilitated by a local router or hub 304 or may be a peer-to-peer mesh; in either case, the communication interface 230 of each cobot enables real-time exchange of information. Through this network, the cobots share sensor data and state information to form a unified understanding of the environment, creating a collective perception. For instance, cobot 100a positioned near the operating table can relay its camera feed of the surgical field to cobot 100b, which might be across the room preparing instruments, so that 100b is aware of the ongoing procedure’s status. Likewise, cobot 100b can transmit data about the status of instrument reprocessing or inventory levels to others. This constant data sharing means each cobot is not limited to its own sensors; rather, it benefits from the sensing capabilities of the entire group, resulting in a more holistic situational awareness for all units.

In addition to sharing raw and processed sensor inputs, the networked system of FIG. 3 enables dynamic coordination of movement and tasks among the cobots. The communication link 302 carries messages such as each cobot’s current position, planned path, and task execution status. With this information, the cobots can adjust their paths in real time to avoid interfering with one another or with humans, effectively traffic-managing themselves in the constrained space of an operating theater. For example, if cobot 100a is transporting a needed surgical tool towards the sterile field and cobot 100c is moving toward the supply cabinet on a potentially intersecting path, they will exchange positions and trajectories; one unit may slow down slightly or yield to let the other pass first, preventing any impasse or collision. The system can also elect or designate one cobot as a temporary coordinator for specific situations (or utilize a distributed algorithm) to ensure movements are sequenced optimally. Moreover, task sharing is facilitated by this inter-cobot communication: if one cobot detects a new task or a sudden change (say, an urgent need to suction fluid on the floor), it can broadcast a task request to the others. A cobot that is free or better positioned (for instance, one that is already near the area of interest) can volunteer or be assigned to handle it, while the others adjust their own task queues accordingly. In essence, FIG. 3 shows the networked cobotic system behaving as an integrated whole, where each cobot remains autonomous but cooperates through high-speed data exchange to achieve a coordinated, efficient workflow. This distributed architecture avoids any single point of failure or processing bottleneck; even if a central server or one cobot goes offline, the remaining units can continue to communicate peer-to-peer and maintain operational continuity in the perioperative setting.

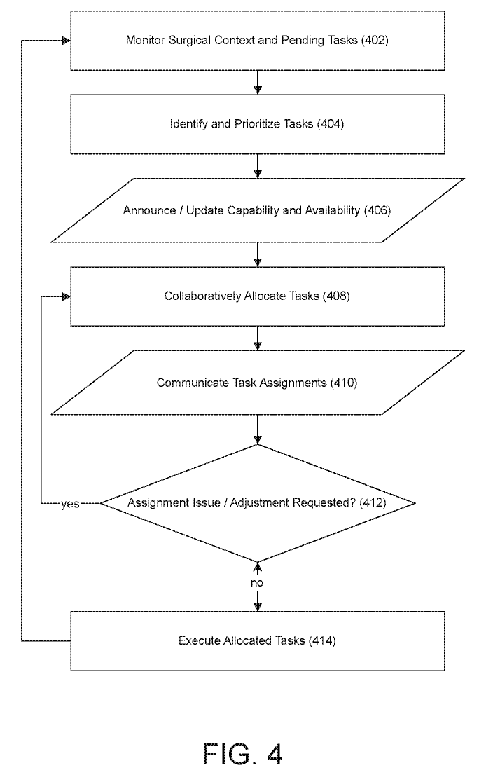

FIG. 4 provides a flowchart of an exemplary collaborative task allocation process as implemented by the system. This process may be executed continuously or in discrete cycles during an operation to dynamically assign and update tasks among the multiple cobots. Initially, the system monitors the surgical context and pending tasks (step 402). This involves gathering inputs such as the current phase of the surgery, outstanding requirements (e.g. an instrument to be delivered, a surface needing decontamination, a call for additional supplies), and each cobot’s status (location, availability, and current assignments). These inputs can come from direct sensor observations, integrated hospital workflow systems, or commands entered by staff. Next, the system identifies and prioritizes tasks (step 404) by analyzing the collected information. For example, tasks might be ranked by urgency: delivering a sterile instrument needed immediately would outrank a routine floor scanning for cleanliness. The system also uses predictive analytics at this stage, leveraging historical data or machine learning models, to anticipate upcoming needs (for instance, predicting that a certain instrument will be needed soon based on the procedure’s progress, or that an interim cleaning will be required in a few minutes).

In the next step, each cobot (through its task management module 250) announces or updates its capability and availability (step 406) to the group. This can include data like: “Cobot 100a is free and has a tool-handling gripper attached,” or “Cobot 100b is currently busy transporting specimens, ETA 2 minutes to free up,” or “Cobot 100c has low battery and will need to recharge soon.” With knowledge of the active tasks and each unit’s state, the system then performs collaborative allocation of the tasks (step 408). In one embodiment, a designated coordination module, which could reside on one cobot or be distributed, runs an optimization algorithm that matches tasks to cobots. This algorithm takes into account factors such as which cobot is nearest to the task location, which has the appropriate tools or sensors, how much workload each currently has, and even the cobots’ learned performance metrics (for example, if one cobot has historically completed a certain type of task faster or more accurately, the system may favor that unit for similar tasks). The allocation can also consider collective efficiency, for instance, grouping tasks so that one cobot handles all instrument deliveries needed in the next 5 minutes while another focuses on continuous monitoring duties.

Once a tentative assignment is made, the system proceeds to communicate the task assignments to the respective cobots (step 410). This may happen via a broadcast message that includes all pairings (e.g., “Cobot A: deliver item X to location Y; Cobot B: prepare instrument Z; Cobot C: standby”) or through direct messaging to each cobot with its specific instruction. The cobots, upon receiving their assignments, may send acknowledgments or, if a cobot detects an issue with its assignment, respond with a negotiation or adjustment request (step 412). For example, if cobot 100c was assigned a task but its battery is low or it detects it cannot reach the location in time, it might decline or request help, prompting the system to reassign that task to another cobot or adjust parameters (perhaps splitting the task or scheduling a short delay if acceptable). Such negotiation ensures that the plan is robust against individual unit constraints. After confirmations, each cobot executes its allocated task (step 414). During execution, they continue to update the group on progress and any new observations. The flowchart is iterative and loops continuously, meaning that as soon as tasks are completed or new tasks arise, the system returns to monitoring (step 402) and re-evaluates the task distribution. This continuous feedback loop enables the team of cobots to adapt in real-time to the dynamic perioperative environment. In summary, FIG. 4’s process ensures that at any given moment, the collective resources of the cobotic system are optimally deployed: workload is balanced, high-priority tasks are addressed promptly by the most suitable unit, and the overall surgical workflow proceeds efficiently with minimal idle time or bottlenecks.

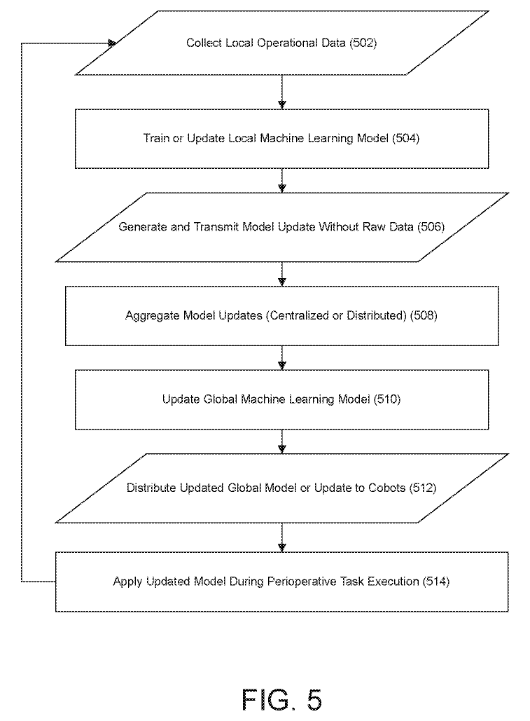

FIG. 5 is a flowchart illustrating an exemplary federated and distributed machine learning workflow executed by a networked cobotic system in a perioperative environment. The flowchart depicts how multiple collaborative robotic devices locally train machine learning models using operational data generated during perioperative activities, exchange model updates without sharing raw data, and iteratively improve a shared or global model while preserving data privacy, reducing network load, and maintaining regulatory compliance. The process shown in FIG. 5 may be performed continuously or periodically during operation of the system and may be implemented in software, firmware, hardware, or combinations thereof on the cobots and associated computing infrastructure.

At Step 502, each cobot collects local operational data generated during performance of perioperative tasks. Such data may include, by way of example and not limitation, sensor data, imaging data, task execution metrics, environmental context data, workflow timing information, detected anomalies, or outcomes of prior task allocations. Importantly, the collected data remains local to the cobot and is not transmitted to other cobots or external systems in raw form.

At Step 504, each cobot trains or updates a local machine learning model using the locally collected operational data. The local model may comprise one or more neural networks, statistical models, reinforcement learning policies, or other machine learning structures configured to support functions such as workflow prediction, instrument recognition, task prioritization, motion planning, or anomaly detection. Local training may occur continuously in the background, during idle periods, or at predefined intervals based on system state or resource availability.

At Step 506, each cobot generates and transmits a model update derived from its locally trained machine learning model. The model update may include, for example, updated model parameters, weight adjustments, gradients, compressed representations, or other learned values sufficient to convey learning progress without exposing the underlying raw data. In some embodiments, the model updates are encrypted, anonymized, or otherwise protected prior to transmission.

Critically, Step 506 is performed without transmitting raw patient data or raw operational data, thereby preserving patient privacy and complying with data protection and healthcare regulatory requirements. The model updates may be transmitted directly to peer cobots, to a designated aggregation entity, or through a combination of centralized and decentralized communication paths.

At Step 508, the system aggregates the model updates received from multiple cobots to produce an aggregated or global model update. Aggregation may be performed by a centralized aggregation node, by one of the cobots acting temporarily as an aggregator, or through a fully distributed peer-to-peer consensus mechanism. The aggregation process may include averaging, weighted combination, or other fusion techniques that account for factors such as data volume, confidence levels, task relevance, or historical performance of individual cobots.

At Step 510, the aggregated model update is applied to update a global machine learning model representing the collective learning of the cobotic system. The global model may be stored locally on each cobot, cached transiently, or maintained in a shared logical state across the networked cobots, depending on system configuration.

At Step 512, the updated global model, or a corresponding global model update, is distributed back to the cobots participating in the federated learning process. Each cobot may then incorporate the updated global model into its local inference and decision-making processes.

At Step 514, each cobot applies the updated model during subsequent perioperative task execution, thereby benefiting from the collective experience of the cobotic system. The process then returns to Step 502, forming a continuous or iterative learning loop in which the system progressively improves performance over time while maintaining data locality and privacy.

The federated learning workflow illustrated in FIG. 5 enables continuous, system-wide learning across multiple cobots without creating centralized data bottlenecks or privacy risks. By exchanging only model updates rather than raw data, the system reduces communication bandwidth requirements, improves fault tolerance, and allows learning to occur even when network connectivity is intermittent. Additionally, the distributed nature of the workflow allows the cobotic system to adapt to new procedures, environments, and operational patterns in real time, enhancing robustness and scalability in complex perioperative settings.

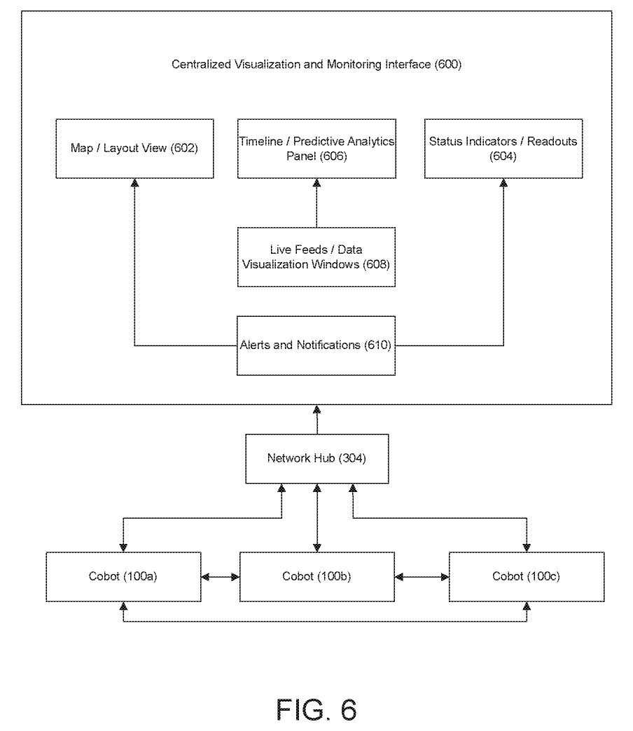

FIG. 6 shows a centralized visualization and monitoring interface 600 for overseeing the cobotic system’s operation in real time. This interface may be a software dashboard accessible on a central workstation in the operating theater or a nurse’s station, or on a mobile device or tablet carried by perioperative staff. The interface 600 aggregates and displays key information from all networked cobots in an intuitive format to facilitate situational awareness and human supervisory control. For example, the interface can include a map or layout view 602 of the operating room or perioperative area, on which the positions of each cobot 100a, 100b, 100c are indicated (perhaps by icons or avatars). This view updates in real time as the cobots move, providing the staff with a clear picture of robot locations relative to the patient, staff, and equipment. The interface 600 also presents status indicators or readouts 604 for each cobot – such as current task description (“transporting instrument tray to OR table”), battery level, mode (active/idle/charging), and any warnings (e.g., “obstacle detected” or “awaiting sterilization clearance”). A timeline or workflow panel 606 may display predictive analytics outcomes: for instance, the system might show anticipated upcoming tasks or alerts (like “Instrument X likely needed in 5 minutes” or “Sterile field contamination risk if current activity continues beyond 10 minutes”) based on the models described earlier. This allows the surgical team to prepare or intervene proactively. The interface can further include live feeds or data visualization windows 608 – for example, showing the video feed from a cobot’s camera when it’s monitoring the surgical field, or graphs of bioburden levels measured over time by a cobot in charge of environmental monitoring. Alerts and notifications 610 are prominently displayed on the interface whenever a safety threshold is crossed or a notable event occurs (such as a cobot pausing due to a person stepping into its path, or a task delay that might impact surgery timing). The centralized interface 600 may also allow limited control inputs from authorized personnel: for instance, a staff member could reassign a task by dragging a task icon from one cobot to another, or could press a “pause all” emergency button on the interface to immediately halt all cobots in an urgent situation. In normal operation, however, the interface primarily serves as an informational and supervisory tool – the cobots autonomously manage their duties, and the surgical team can simply glance at the dashboard to obtain reassurance that everything is proceeding correctly. Overall, FIG. 6 illustrates how the system provides perioperative personnel with comprehensive, real-time oversight of the cobotic assistants’ collective status, activities, and even predictive insights, thereby building trust and enabling effective human oversight without requiring constant manual control of each robot.

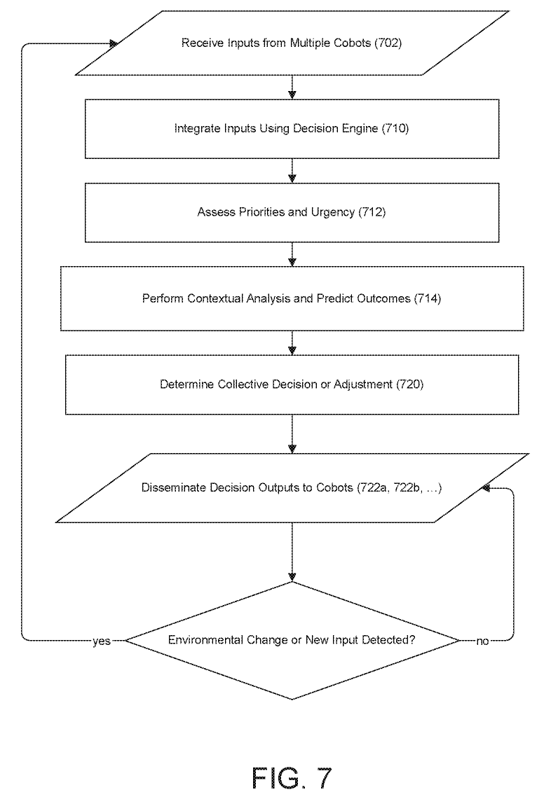

Moving on to collective decision-making, FIG. 7 depicts an adaptive collective decision-making logic 700 employed by the cobots to respond to changing conditions as a coordinated unit. This logic can be understood as a high-level control layer that takes inputs from multiple cobots (and potentially external sources) and produces decisions or adjustments that guide the group’s behavior. As shown schematically in FIG. 7, inputs 702 from various cobots are fed into a decision engine 710. These inputs can include each cobot’s local observations (e.g., sensor detections of events or anomalies), status reports (task progress, any issues encountered), and even their individually computed recommendations or intended actions. For example, one cobot might input “instrument tray delivery complete,” another might input “detected spill on floor,” and a third might input a suggested action like “propose immediate cleanup task.” The decision engine 710 integrates all these concurrent inputs. In some embodiments, the decision engine 710 is a distributed algorithm, meaning each cobot runs a copy of the logic and they arrive at a consensus through message-passing. In other embodiments, one cobot or a central controller might momentarily act as the arbiter for a group decision, especially if quick coordination is needed. Regardless of implementation, the logic is adaptive in that it does not follow a rigid pre-scripted sequence, but rather evaluates the current state and determines an optimal collective response on the fly.

Within the decision engine 710, multiple factors and rules are considered. There may be a priority assessment module 712 that weighs the urgency and importance of the inputs: for instance, a safety-critical input (like detection of a person entering a restricted zone) would be given highest priority in influencing the next actions. There can also be a contextual analysis module 714 which uses the shared knowledge base (potentially enhanced by the learned models from FIG. 5) to predict outcomes of different possible decisions. For example, if faced with an unforeseen event such as a power tool malfunction, the system might predict that pausing certain non-critical tasks to focus all cobots on troubleshooting or containing the issue is the best course. The decision-making logic could employ techniques like voting schemes, weighted consensus (where a cobot’s confidence level in its suggestion affects its influence), or even a trained collective policy model that was developed through machine learning. Once a collective decision is reached (block 720), it is disseminated to the individual cobots as updated directives. FIG. 7 illustrates this as decision output signals 722a, 722b going to cobot 100a and 100b, etc., instructing them on their roles in the decided course of action. An example scenario of the adaptive collective decision-making in practice: suppose during a surgery, an unexpected complication arises that requires immediate blood transfusion and additional instruments. The cobots’ sensors and possibly integration with the hospital system recognize this change; one cobot’s input might flag “urgent transfusion kit needed,” another flags “surgery extended, prepare additional instrument set.” The decision logic 700 collectively decides to reprioritize tasks: perhaps diverting a cobot that was doing routine cleaning to instead fetch the transfusion kit, while another cobot extends its monitoring tasks. This new plan is communicated to all units so they each understand the adjusted roles and can act without confusion or conflict. As the situation continues to evolve, the logic will adapt further – for instance, if the transfusion kit is delivered and the urgency subsides, the cobots can smoothly transition back to their prior routine or to a new equilibrium of tasks. The adaptive collective decision-making of FIG. 7 thus enables the cobotic system to collectively respond to unforeseen events or dynamic changes in the perioperative environment, ensuring that the group’s actions remain synchronized with the immediate needs and goals of the surgical procedure. It effectively endows the robot team with a form of group intelligence or hive mind for high-level decisions, while still allowing each cobot to carry out its individual actions in concert with the others.

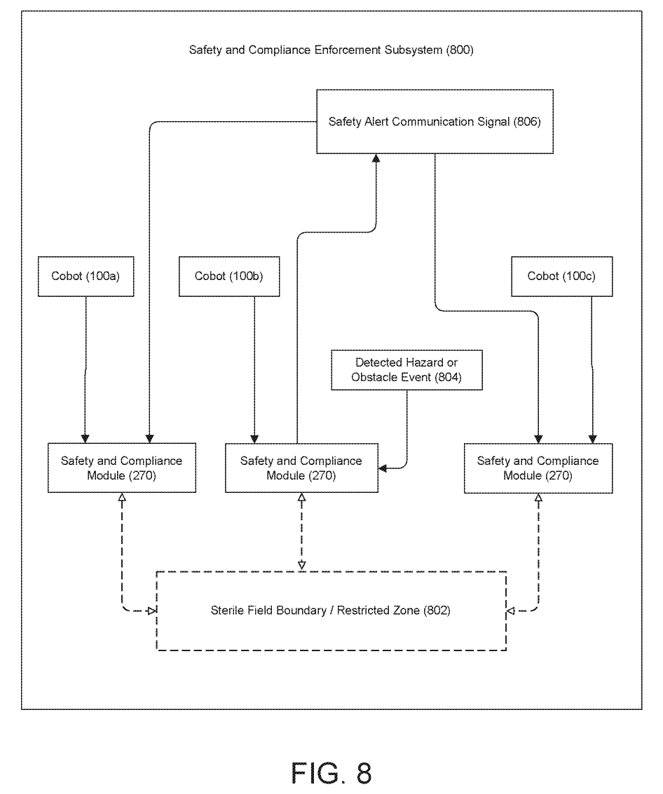

Finally, FIG. 8 illustrates a self-organizing safety and compliance enforcement subsystem 800 that underpins the safe operation of the cobotic team. In the diagram, multiple cobots 100 are shown collaboratively maintaining safety constraints and protocol compliance without requiring centralized oversight for every rule. Each cobot is equipped with its local safety/compliance module 270 (from FIG. 2), and FIG. 8 emphasizes how these modules interact through inter-cobot communication to form a collective safety network. One aspect of this subsystem is the enforcement of sterile field boundaries and other no-go zones. For example, a sterile field or restricted zone around the patient may be defined 802. Each cobot continuously tracks its own position relative to such boundaries using on-board sensors (like machine vision to recognize drapes or floor markings, and spatial data from the navigation module 240). If a cobot needs to hand over an instrument or perform a task near the sterile field, it will do so in a compliant manner (e.g., stopping at the boundary and using an extendable arm to pass an instrument without driving into the sterile area). The subsystem 800 ensures coordinated adherence to this by having the cobots share their intentions and movements near the boundary. For instance, before cobot 100a extends into the sterile zone, it might send a notice to others that it is performing a sterile handoff, so all other cobots give it a wide berth and hold any movements that could accidentally jostle it at that time.

Another key facet depicted in FIG. 8 is obstacle and human avoidance through self-organization. Each cobot’s safety module 270 processes sensor data to detect humans (surgeons, nurses, etc.) or unexpected obstacles (spilled liquids, dropped tools, etc.) in real time. When one cobot (say 100b) detects a person stepping into its path or a new obstacle on the floor (event 804), it will immediately take local protective action (such as decelerating to a stop, sounding an alert, or rerouting around the person/obstacle). Simultaneously, it sends a communication alert to the network 806 informing all other cobots of the hazard. Upon receiving such an alert, the other cobots autonomously adjust their behavior even if they haven’t yet seen the hazard themselves. For example, cobot 100c might slow its speed preemptively because it knows 100b has encountered a nearby nurse, thereby reducing the likelihood of any surprise encounter. In this way, safety information propagates instantly through the group, and the response is self-organizing: there is no need for a central controller to instruct each unit to behave safely; instead each unit independently takes appropriate action that aligns with the others because they all share the same safety rules and communicate their states. This collective behavior extends to yielding and route negotiation, if two cobots approach a narrow corridor at the same time, their safety systems, communicating, will cause one to yield and wait while the other passes, according to pre-established rules or priority criteria (for instance, whichever is currently on a critical time-sensitive task may get right-of-way).

The subsystem 800 also covers regulatory compliance and fail-safe procedures. All cobots continuously monitor for any operation that might violate hospital protocols (such as entering a restricted area, failing to sanitize after certain tasks, or operating in a manner outside approved parameters). If any such condition is detected by a cobot, it will not only correct its own behavior (e.g., immediately halt and flash a warning if it were to inadvertently approach an off-limits area), but also inform the team and the central interface (FIG. 6) so that the issue is logged and brought to human attention if necessary. The term “self-organizing” underscores that the cobots collectively enforce these rules: they share responsibility in monitoring each other to some extent. For instance, if one cobot were to malfunction or lose localization and start moving irregularly, another cobot’s sensors might detect this; the observing cobot can then issue a network command that triggers an emergency stop on the errant unit and perhaps all units, pausing the entire system in a safe state within milliseconds. Each cobot is thereby both a watcher and an actor in the safety enforcement web. Additionally, the cobots are designed to default to safe states automatically in case of any detected anomaly – such as stopping motion, locking out potentially dangerous actuators, or retracting arms – and to broadcast state signals (e.g., “EMERGENCY_PAUSE”) that prompt the rest of the team to also enter a precautionary hold pattern. This self-organizing safety subsystem 800 ensures that even in complex, rapidly changing situations, the collective of cobots upholds the highest safety standards and complies with all operational protocols of the surgical environment. By having multiple robots cross-monitor and adapt to each other’s findings, the system builds in redundancy and fault tolerance; no single point of failure can easily lead to an unsafe condition because another cobot is likely to catch and compensate for it. In effect, FIG. 8 highlights that safety and compliance are ingrained at every level of the cobotic system, from the individual robot’s instant reflexes to the group’s coordinated protective maneuvers, all happening autonomously and in real-time without direct human micromanagement.

Through the detailed descriptions of FIGS. 1–8 above, it can be appreciated how the various subsystems and processes interoperate to achieve the collective perioperative functionality of the invention. Each cobot is structurally and functionally equipped to perform autonomous tasks and make intelligent decisions (FIGS. 1–2), and when multiple such cobots are networked together (FIG. 3), they leverage inter-cobot communication to function as a cohesive team. The collaborative task allocation mechanisms (FIG. 4) ensure efficient division of labor in real time, while the distributed learning framework (FIG. 5) means the team as a whole becomes more skilled and adaptive with experience. The centralized interface (FIG. 6) keeps human operators in the loop by providing oversight and predictive insights without requiring continuous intervention. The adaptive decision logic (FIG. 7) allows the robotic team to dynamically adjust to new conditions or requirements as a unified entity. Finally, the self-organizing safety and compliance subsystem (FIG. 8) underlies all operations, guaranteeing that the pursuit of efficiency and learning never compromises safety or protocol adherence. In combination, these elements support the claimed invention’s ability to enhance perioperative functionality through distributed intelligence and collaboration, enabling a smarter, safer, and more effective robotic presence in the surgical theater.

While specific embodiments have been described to enable practice of the disclosed subject matter, they are illustrative and not limiting. Variations, modifications, substitutions, and combinations of the features described can be made by those skilled in the art without departing from the scope of this disclosure. Features described with respect to one embodiment may be used with, or substituted for, features of another. Unless expressly stated otherwise, the terms “comprising,” “including,” and “having” are used in an open, non‑limiting sense; the singular forms “a,” “an,” and “the” include the plural forms; and stated ranges include their endpoints and all subranges. Unless the context clearly indicates otherwise, “and/or” means one or more of the listed items (e.g., “A and/or B” encompasses A alone, B alone, or both A and B); “or” is inclusive (i.e., “A or B” means A, B, or both); and “and” is conjunctive. Headings and ordering are for convenience and do not limit the description. The scope of the claimed subject matter is defined solely by the claims that follow and their legal equivalents.

What is claimed is:

1. A system for distributed learning and inter-cobot collaboration in a perioperative environment, the system comprising:
a plurality of cobots configured to operate in the perioperative environment, each cobot including at least one processor, at least one memory, and a communication interface for exchanging data with other cobots, wherein the plurality of cobots are configured to collectively:
exchange data in real time via the communication interfaces to share information and coordinate cobot actions;
perform a federated learning process in which each cobot trains a local machine learning model using data collected by that cobot and shares model updates with the other cobots to collaboratively improve a global machine learning model without sharing raw data, thereby preserving patient privacy; and
dynamically allocate and execute perioperative tasks among the cobots based on real-time situational assessments of the perioperative environment and predictive analytics, such that the cobots share tasks, exchange data, and learn collectively without compromising patient privacy.

2. The system of claim 1, wherein the communication interface of each cobot comprises a wireless, low-latency network link that enables direct peer-to-peer communication among the cobots for real-time coordination.

3. The system of claim 1, wherein each collaborative robot further comprises one or more sensors to monitor the perioperative environment, and wherein the situational assessment used for task allocation is based on sensor data from the one or more sensors of the cobots.

4. The system of claim 1, wherein the predictive analytics include applying a machine learning model trained on historical perioperative data to predict upcoming procedure phases or resource needs, and the collaborative task allocation accounts for these predicted procedure phases in distributing tasks among the cobots.

5. The system of claim 1, wherein each cobot is configured to share only processed model parameters or updates during the federated learning process and not raw patient data, such that each cobot’s local data remains private.

6. The system of claim 1, wherein the collaborative task allocation is based at least in part on each cobot’s current operational status and location, assigning tasks to cobots according to their capabilities and proximity to where each task is to be performed.

7. The system of claim 1, wherein the cobots are configured to collectively maintain compliance with sterile field protocols by sharing their positions with one another and coordinating movements to avoid breaching a defined sterile boundary during a surgical procedure.

8. The system of claim 1, further comprising a user interface in communication with the cobots, the user interface being configured to display real-time status information, task assignments, and alerts regarding the cobots’ operations to perioperative personnel for centralized monitoring of the collaborative robotic system.

9. A computer-implemented method for enhanced perioperative collaboration using a plurality of collaborative robotic devices, the method comprising:
establishing a communication link among the collaborative robotic devices to enable real-time inter-cobot data exchange;
collecting local operational data at each collaborative robotic device and training a local machine learning model on the collected data;
transmitting, by each collaborative robotic device via the communication link, model updates derived from the local machine learning model to one or more other collaborative robotic devices;
aggregating the model updates from the collaborative robotic devices to update a global machine learning model without transferring the underlying raw data, thereby preserving patient privacy;
analyzing a current situational state of the perioperative environment using data shared among the collaborative robotic devices, wherein the current situational state of the perioperative environment includes collaborative robotic device utilization;
predicting, based on the analyzed situational state and historical procedure data, one or more future task requirements or changes in the perioperative environment using predictive analytics;
determining, based on the analyzed situational state and the predicted future requirements, an allocation of perioperative tasks among the collaborative robotic devices; and
executing, by each of the collaborative robotic devices, its allocated tasks in a coordinated manner via the communication link to carry out the tasks collectively.

10. The method of claim 9, wherein establishing the communication link comprises setting up a wireless ad-hoc network among the collaborative robotic devices with a latency below a predetermined threshold to support real-time coordinated behavior.

11. The method of claim 9, wherein analyzing the current situational state includes processing sensor data from the collaborative robotic devices, the sensor data comprising information from imaging sensors, proximity sensors, and vital sign monitors to determine contextual information about the ongoing surgical procedure.

12. The method of claim 9, wherein the predictive analytics comprises applying a trained machine learning model to historical and real-time perioperative data to forecast upcoming procedural steps and potential resource bottlenecks.

13. The method of claim 12, wherein determining the allocation of tasks includes adjusting task assignments for the collaborative robotic devices in anticipation of the forecasted procedural steps, such that each device is preemptively assigned tasks to address predicted needs in the surgical workflow.

14. The method of claim 9, further comprising, in response to detecting an unexpected change in the perioperative environment or a disruption in the surgical workflow, dynamically reassigning at least one of the tasks among the collaborative robotic devices to adapt to the change in real time.

15. The method of claim 9, wherein transmitting the model updates includes encrypting the model updates and performing a secure aggregation of the updates so that no individual device’s raw data is exposed to any other device during the federated learning process.

16. One or more non-transitory computer-readable media storing instructions that, when executed by one or more processors of a system comprising a plurality of collaborative robotic devices operating in a perioperative environment, cause the system to:
establish a communication network among the collaborative robotic devices to enable real-time data exchange for coordinated operation;
collect operational data at each collaborative robotic device and train a local machine learning model on the collected data;
transmit model updates from each collaborative robotic device to other collaborative robotic devices over the communication network, the model updates being derived from the local machine learning model;
aggregate the model updates from the plurality of devices to update a global machine learning model without transmitting raw operational data, thereby preserving the privacy of patient-related data;
analyze a current state of the perioperative environment based on data shared among the collaborative robotic devices;
utilize predictive analytics on the analyzed state to anticipate future task needs or changes in the perioperative environment;
determine an allocation of perioperative tasks among the collaborative robotic devices based on the current state and anticipated future needs; and
control the collaborative robotic devices to execute their allocated tasks in coordination with one another, thereby enabling the devices to share tasks, exchange data, and learn collectively without compromising patient privacy.

17. The one or more non-transitory computer-readable media of claim 16, wherein the perioperative tasks allocated by the instructions comprise at least one of: instrument delivery to a surgeon, surgical site retraction or stabilization, patient vital sign monitoring, collaborative robotic device monitoring, and sterile field management during a procedure.

18. The one or more non-transitory computer-readable media of claim 16, wherein the instructions further cause the one or more processors to, upon detecting an anomalous condition or an interruption in the surgical workflow, automatically adjust the task allocation by reassigning one or more tasks among the collaborative robotic devices to accommodate the change.

19. The one or more non-transitory computer-readable media of claim 16, wherein the instructions cause the system to transmit data describing the status and activities of the collaborative robotic devices to a display interface in real time, providing perioperative personnel with a unified view of the cobots’ operations and alerts.

20. The one or more non-transitory computer-readable media of claim 16, wherein the instructions cause each collaborative robotic device to broadcast its real-time position and planned trajectory to the other devices via the communication network and to modify its own trajectory in response to trajectories received from the other devices, thereby avoiding collisions and interference among the cobots during task execution.
